(19) United States
(12) Patent Application Publication
(10) Pub. No.: US 2024/0083883 A1
(43) Pub. Date: Mar. 14, 2024

(54) RAD51 BINDING INHIBITORS AND METHODS OF USE THEREOF

(71) Applicant: Sarissa Inc., London (CA)

(72) Inventors: Mark David VINCENT, London (CA); Peter John Ferguson, London (CA); Donald James Koropatnick, London (CA)

(21) Appl. No.: 18/270,624

(22) PCT Filed: Dec. 31, 2021
(86) PCT No.: PCT/CA2021/051906

§ 371 Date: Jun. 30, 2023

Related U.S. Application Data
(60) Provisional application No. 63/132,833, filed on Dec. 31, 2020.

(51) Int. Cl. C07D 403/14; A61K 31/138; A61K 31/4166; A61K 31/44; A61K 31/506; A61K 31/517; A61P 35/00; C07D 209/44; C07D 401/10; C07D 471/04

(52) U.S. Cl. CPC C07D 403/14; A61K 31/138; A61K 31/4166; A61K 31/44; A61K 31/506; A61K 31/517; A61P 35/00; C07D 209/44; C07D 401/10; C07D 471/04

(57) ABSTRACT

The present invention provides RAD51 inhibitors having the structural formula (I): or a pharmaceutically acceptable salt or solvate thereof. Also provided are methods of treating or preventing cancer comprising administration of the compounds of the present invention, as well as uses of the compounds to induce a synergistic effect with known chemotherapeutic.

### FIELD OF THE INVENTION

The present invention pertains to the field of cancer therapeutics, and in particular to compounds that inhibit RAD51.

### BACKGROUND

Double-strand breaks (DSBs) in DNA are considered the principal lethal damage resulting from irradiation and/or exposure to cross-linking drugs (e.g., cisplatin, mitomycin, doxorubicin, bleomycin, etc.). Typically, DSBs lead to arrest in the cell cycle progression and to activation of the cellular DNA repair machinery, and failure of DNA repair generally leads to genome abnormality, and eventually cell death.

RAD51 protein plays an essential role in proliferation and cell viability and genome integrity in cells, as well as in the machinery for repair of DSBs. Cells have two homologous copies of each chromosome. RAD51, guided by BRCA2, associates with one end of a DSB in the damaged chromosome to help the broken strand ‘find’ the other (intact, homologous) chromosome and use it for information essential for repair. RAD51 enables the broken segment to ‘invade’ the intact chromosome, and the DSB in the damaged chromosome is rejoined using information copied from the undamaged homologous chromosome. The repair increases the chance of survival of cells that sustain DSBs in their DNA. The mechanisms by which the RAD51 protein stabilizes the cancer genome leading to its increased resilience are not necessarily fully understood and may also include such phenomena as protection of stalled replication forks and other known mechanisms, in addition to DSB repair.

Although RAD51 is essential for normal cells, cancer cells depend much more on RAD51 for survival because of the greater degree of DNA damage they typically sustain, either spontaneously or as a result of therapy. Overexpression of RAD51 is observed in many cancer cells, facilitating increased repair of DNA damage caused by cancer-related genome instability and exposure to drug treatments and radiotherapy which can, in turn, contribute to the resistance of cancer cells to chemotherapies and lead to lower survival among cancer patients. Overexpression of RAD51 has been observed in cancers of the pancreas, breast, prostate, lung, brain and melanoma.

Blocking interaction of RAD51 with BRCA2 can provide a route for the development of anti-cancer therapeutics by preventing repair of damaged DNA, leading to cumulative DNA damage that is ultimately lethal to the cell.

US Patent Publication 2009/0221634 discloses small molecule inhibitors, including an inhibitor designated as IBR2, which was identified and evaluated for its ability to disrupt BRCA2-RAD51 interactions and inhibit cancer cell growth. Although RAD51 was degraded in IBR2-treated cancer cells, and the homologous recombination repair was impaired, leading to cell death, the IC₅₀ values of IBR2 were only in the range of 12-20 μM for most tested cancer cell lines.

A structurally similar compound, IBR120, was disclosed in US Patent Publication No. 2018/0057483, which was also observed to inhibit RAD51.

Unfortunately, IBR2 and IBR120 have low aqueous solubility, which can lead to low bioavailability, as well as challenges in preparing pharmaceutical formulations.

There is therefore a need for the development of highly water-soluble small molecules that effectively inhibit RAD51 and thus exhibit anti-cancer activity. There is also a need for novel cancer therapeutic agents that can be used in combination with known therapies, and which may exhibit a synergistic effect.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide RAD51 binding inhibitors and methods of use thereof. In accordance with an aspect of the present invention, there is provided novel compounds of structural formula (I):

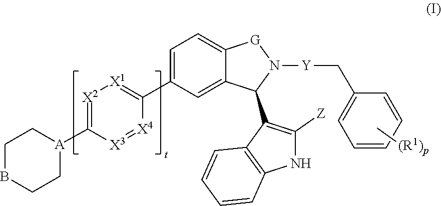

or a pharmaceutically acceptable salt or solvate thereof, wherein: A is N or CH; B is CH₂, O, or NR^{a}, wherein R^{a} is —H or C₁₋₄ alkyl; X¹, X², X³ and X⁴ are each independently N or CR; t is 0 or 1; G is —CH₂—, —CHOH, —CH═CH—, —COH═CH—, or —CH═COH—; Z is H or OH; R is independently hydrogen, halogen, hydroxyl, C₁₋₆ alkyl, C₁₋₆ alkoxyl, or N(R^{b})₂, wherein R^{b} is —H or C₁₋₆ alkyl; Y is —C(O)— or —SO₂—; R¹ is —(CH)ₙX—R²; p is an integer from 0-5; n is an integer from 0-6; X is absent, —C(O)—, —NHC(O)—, —NHC(O)CH₂—, —C(O)CH₂NHC(O)— or —C(O)NH—; and R² is hydrogen, a cycloalkyl group, a heterocyclic group, a heteroaromatic group, or an aryl group, wherein the cycloalkyl group, heterocyclic group, heteroaromatic group, and aryl group are optionally substituted with one or more substituents selected from halogen, hydroxyl, oxo, and lower alkoxyl.

In accordance with another aspect of the present invention, there is provided a method of treating or preventing cancer in a subject in need thereof, the method comprising the step of administering a therapeutically effective amount of a compound in accordance with the present invention, or a pharmaceutically acceptable salt or solvate thereof, to the subject.

In accordance with another aspect of the present invention, there is provided a method of treating or preventing cancer in a subject in need thereof, the method comprising the step of administering a therapeutically effective amount of a compound in accordance with the present invention, or a pharmaceutically acceptable salt or solvate thereof, in combination with a chemotherapeutic agent, to the subject.

In accordance with another aspect of the present invention, there is provided use of a therapeutically effective amount of a compound in accordance with the present invention, or a pharmaceutically acceptable salt or solvate thereof, for the treatment or prevention of cancer in a subject in need thereof.

In accordance with another aspect of the present invention, there is provided use of a therapeutically effective amount of a compound in accordance with the present invention, or a pharmaceutically acceptable salt or solvate thereof, in combination with a chemotherapeutic agent, for the treatment or prevention of cancer in a subject in need thereof.

In accordance with another aspect of the present invention, there is provided use of a compound in accordance with the present invention, or a pharmaceutically acceptable salt or solvate thereof, to reduce the effective therapeutic dose of a chemotherapeutic agent for the treatment or prevention of cancer in a subject in need thereof.

In accordance with another aspect of the present invention, there is provided use of a combination of a chemotherapeutic agent and a compound in accordance with the present invention, or a pharmaceutically acceptable salt or solvate thereof, to reduce the effective therapeutic dose of the compound for the treatment or prevention of cancer in a subject in need thereof.

### BRIEF DESCRIPTION OF THE FIGURES

FIG. 1a is a ¹H-NMR spectrum of Compound 1, JKYN-1 (base), in accordance with one embodiment of the present invention.

FIG. 1b is a ¹H-NMR spectrum of JKYN-1-mesylate, in accordance with one embodiment of the present invention.

FIG. 2 is a graph depicting the inhibitory effect of JKYN-1-mesylate, as a single agent, on the growth of NSCLC cell line H1650.

FIG. 3A is a graph depicting the effect of JKYN-1-mesylate on inhibition by osimertinib of the growth of NSCLC cell line H1650.

FIG. 3B is a graph depicting the effect of osimertinib on inhibition by JKYN-1-mesylate of the growth of NSCLC cell line H1650.

FIGS. 4A-C are a series of bar graphs depicting the synergistic cytotoxic effect of the combination of JKYN-1-mesylate and osimertinib on inhibiting the growth of NSCLC cell line H1650.

FIG. 5 is a graph depicting the inhibitory effect of JKYN-1-mesylate, as a single agent, on the growth of prostate carcinoma cell line LNCaP.

FIG. 6A is a graph depicting the effect of JKYN-1-mesylate on inhibition by enzalutamide of the growth of prostate carcinoma cell line LNCaP.

FIG. 6B is a graph depicting the effect of enzalutamide on inhibition by JKYN-1-mesylate of the growth of prostate carcinoma cell line LNCaP.

FIGS. 7A-B are a series of bar graphs depicting the synergistic cytotoxic effect of the combination of JKYN-1-mesylate and enzalutamide on inhibiting the growth of prostate carcinoma cell line LNCaP.

FIG. 8 is a graph depicting the inhibitory effect of JKYN-1-mesylate, as a single agent, on the growth of MCF-7 breast carcinoma cell line.

FIG. 9A is a graph depicting the effect of JKYN-1-mesylate on inhibition by 4-OH-tamoxifen of the growth of MCF-7 breast carcinoma cell line.

FIG. 9B is a graph depicting the effect of 4-OH-tamoxifen on inhibition by JKYN-1-mesylate of the growth of MCF-7 breast carcinoma cell line

FIGS. 10A-C are a series of bar graphs depicting the synergistic cytotoxic effect of the combination of JKYN-1-mesylate and 4-OH-tamoxifen on inhibiting the growth of MCF-breast carcinoma cell line.

FIG. 11 is a graph depicting the inhibitory effect of JKYN-1-mesylate, as a single agent, on the growth of N87 stomach carcinoma cell line.

FIG. 12A is a graph depicting the effect of JKYN-1-mesylate on inhibition by regorafenib of the growth of N87 stomach carcinoma cell line.

FIG. 12B is a graph depicting the effect of regorafenib on inhibition by JKYN-1-mesylate of the growth of N87 stomach carcinoma cell line

FIG. 13 is a bar graph depicting the synergistic cytotoxic effect of the combination of JKYN-1-mesylate and regorafenib on inhibiting the growth of N87 stomach carcinoma cell line.

FIG. 14 is a graph depicting the inhibitory effect of JKYN-1 (base), JKYN-1-mesylate and prior art IBR120, each employed as a single agent, on the growth of NSCLC cell line A549b.

FIG. 15 is a graph depicting the inhibitory effect of JKYN-1 (base), JKYN-1-mesylate and prior art IBR120, each employed as a single agent, on the growth of pancreatic cell line PANC-1.

FIG. 16A is a graph depicting the effect of JKYN-1-mesylate on inhibition by enzalutamide of the growth of DU145 prostate carcinoma cell line.

FIG. 16B is a graph depicting the effect of enzalutamide on inhibition by JKYN-1-mesylate of the growth of DU145 prostate carcinoma cell line.

FIG. 17 is a graph depicting the change in the IC₅₀ values of JKYN-1-mesylate in the presence of increasing amounts of enzalutamide in DU145 prostate carcinoma cell line assay.

FIG. 18 is a bar graph depicting the synergistic cytotoxic effect of the combination of JKYN-1-mesylate and enzalutamide on inhibiting the growth of DU145 prostate carcinoma cell line.

FIG. 19 is a graph depicting the effect of JKYN-1-mesylate on inhibition by regorafenib of the growth of NSCLC cell line A549b.

FIG. 20 is a graph depicting the effect of JKYN-1-mesylate on inhibition by afatinib of the growth of PANC-1 cell line.

FIG. 21 is a graph depicting the reduction in the IC₅₀ values of JKYN-1-mesylate in the presence of increasing amounts of osimertinib in H1650 NSCLC cell line assay.

FIG. 22 is a graph depicting the reduction in the IC₅₀ values of JKYN-1-mesylate in the presence of increasing amounts of 4-OH-tamoxifen in MCF-7 breast carcinoma cell line assay.

FIG. 23 is a graph depicting the effect of JKYN-1 on normal cell line surrogates.

### DETAILED DESCRIPTION OF THE INVENTION

The present invention relates in part to the discovery of novel RAD51 inhibitors that are useful for the treatment or prevention of cancer. As demonstrated herein, the compounds of the present invention have been shown to be effective chemotherapeutic agents for the treatment of cancer. The compounds of the present invention have also demonstrated a synergistic effect when administered in combination with known chemotherapeutic agents.

The present invention provides novel compounds of structural formula (I):

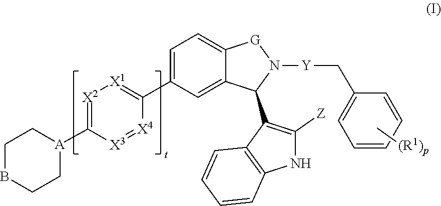

or a pharmaceutically acceptable salt or solvate thereof, wherein:
A is N or CH;
B is CH₂, O, or NR^{a}, wherein R^{a} is —H or C₁₋₄ alkyl;
X¹, X², X³ and X⁴ are each independently N or CR;
t is 0 or 1;
G is —CH₂—, —CHOH, —CH═CH—, —COH═CH—, or —CH═COH—;
Z is H or OH;
R is independently halogen, hydroxyl, C₁₋₆ alkyl, C₁₋₆ alkoxyl, or N(R^{b})₂, wherein R^{b} is —H or C₁₋₆ alkyl;
Y is —C(O)— or —SO₂—;
R¹ is —(CH)ₙX—R²;
p is an integer from 0-5;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the cycloalkyl group, heterocyclic group, heteroaromatic group, and aryl group are optionally substituted with one or more substituents selected from halogen, hydroxyl, oxo, and lower alkoxyl.

In one embodiment, the present invention provides novel compounds of structural formula (Ia):

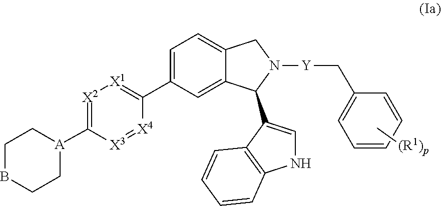

or a pharmaceutically acceptable salt or solvate thereof, wherein:
A is N or CH;
B is CH₂, O, or NR^{a}, wherein R^{a} is —H or C₁₋₄ alkyl;
X¹, X², X³ and X⁴ are each independently N or CR;
R is independently hydrogen, halogen, hydroxyl, C₁₋₆ alkyl, C₁₋₆ alkoxyl, or N(R^{b})₂, wherein R^{b} is —H or C₁₋₆ alkyl;
Y is —C(O)— or —SO₂—;
R¹ is —(CH)ₙX—R²;
p is an integer from 0-5;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the cycloalkyl group, heterocyclic group, heteroaromatic group, and aryl group are optionally substituted with one or more substituents selected from halogen, hydroxyl, oxo, and lower alkoxyl.

In one embodiment, the present invention provides novel compounds of structural formula (Ib):

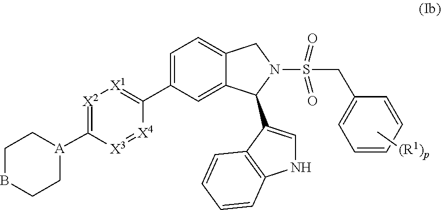

or a pharmaceutically acceptable salt or solvate thereof, wherein:
X¹, X², X³ and X⁴ are each independently selected from N and CH;
A is N or CH;
B is CH₂, O, or NR^{a}, wherein R^{a} is —H or C₁₋₄ alkyl;
X¹, X², X³ and X⁴ are each independently N or CR;
R is independently hydrogen, halogen, hydroxyl, C₁₋₆ alkyl, C₁₋₆ alkoxyl, or N(R^{b})₂, wherein R^{b} is —H or C₁₋₆ alkyl;
R¹ is —(CH)ₙX—R²;
p is an integer from 0-5;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the heterocyclic group, heteroaromatic group, and aryl group are optionally substituted with one or more substituents selected from halogen, hydroxyl, oxo, and lower alkoxyl;

In a further embodiment, the compounds of the present invention have the structural formula (II):

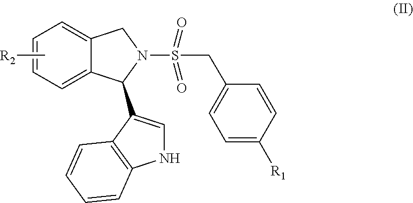

or a pharmaceutically acceptable salt or solvate thereof, wherein:
R₂ is

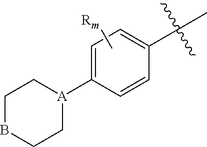

A is N or CH;
B is CH₂, O, or NR^{a}, wherein R^{a} is —H or C₁₋₄ alkyl;
R is independently hydrogen, halogen, hydroxyl, C₁₋₆ alkyl, C₁₋₆ alkoxyl, or N(R^{b})₂, wherein R^{b} is —H or C₁₋₆ alkyl; and
m is an integer from 0-4;
R₁ is —(CH)ₙX—R²;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the heterocyclic group, heteroaromatic group, and aryl group are optionally substituted with one or more substituents selected from halogen, hydroxyl, oxo, and lower alkoxyl.

In a further embodiment, the compounds of the present invention have the structural formula (III):

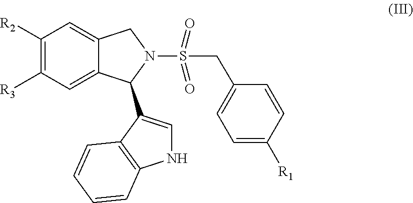

or a pharmaceutically acceptable salt or solvate thereof, wherein:
one of R₂ and R₃ is hydrogen, and the other of R₂ and R₃ is

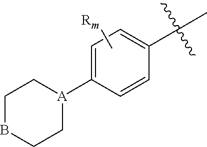

wherein:
A is N or CH;
B is CH₂, O, or NR^{a}, wherein R^{a} is —H or C₁₋₄ alkyl;
R is independently hydrogen, halogen, hydroxyl, C₁₋₆ alkyl, C₁₋₆ alkoxyl, or N(R^{b})₂, wherein R^{b} is —H or C₁₋₆ alkyl; and
m is an integer from 0-4;
R₁ is —(CH)ₙX—R²;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the heterocyclic group, heteroaromatic group, and aryl group are optionally substituted with one or more substituents selected from halogen, hydroxyl, oxo, and lower alkoxyl.

In another embodiment, the compounds of the present invention have the structural formula (IV):

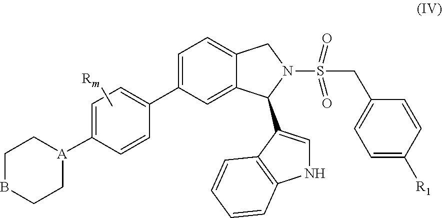

or a pharmaceutically acceptable salt or solvate thereof, wherein:
A is N or CH;
B is CH₂, O, or NR^{a}, wherein R^{a} is —H or C₁₋₄ alkyl;
R is independently hydrogen, halogen, hydroxyl, C₁₋₆ alkyl, C₁₋₆ alkoxyl, or N(R^{b})₂, wherein R^{b} is —H or C₁₋₆ alkyl;
m is an integer from 0-4;
R₁ is —(CH)ₙX—R²;
n is an integer from 0-6;
X is absent, —C(O)—, —NHC(O)—, —NHC(O)CH₂—, —C(O)CH₂NHC(O)— or —C(O)NH—,
R² is hydrogen, a cycloalkyl group, a heterocyclic group, a heteroaromatic group, or an aryl group, wherein the heterocyclic group, heteroaromatic group, and aryl group are optionally substituted with one or more substituents selected from halogen, hydroxyl, oxo, and lower alkoxyl.

In preferred embodiments of the invention, the compounds of formulas (I), (Ia), (Ib), (II), (III) and (IV) are compounds wherein at least one of A is N and B is NR^{a}.

In a further preferred embodiment, the compound is provided in the salt form. In yet a further preferred embodiment, the salt form is the methanesulfonate (or mesylate) salt of the compound.

Exemplary compounds falling within the scope of the present invention include, but are not limited to:

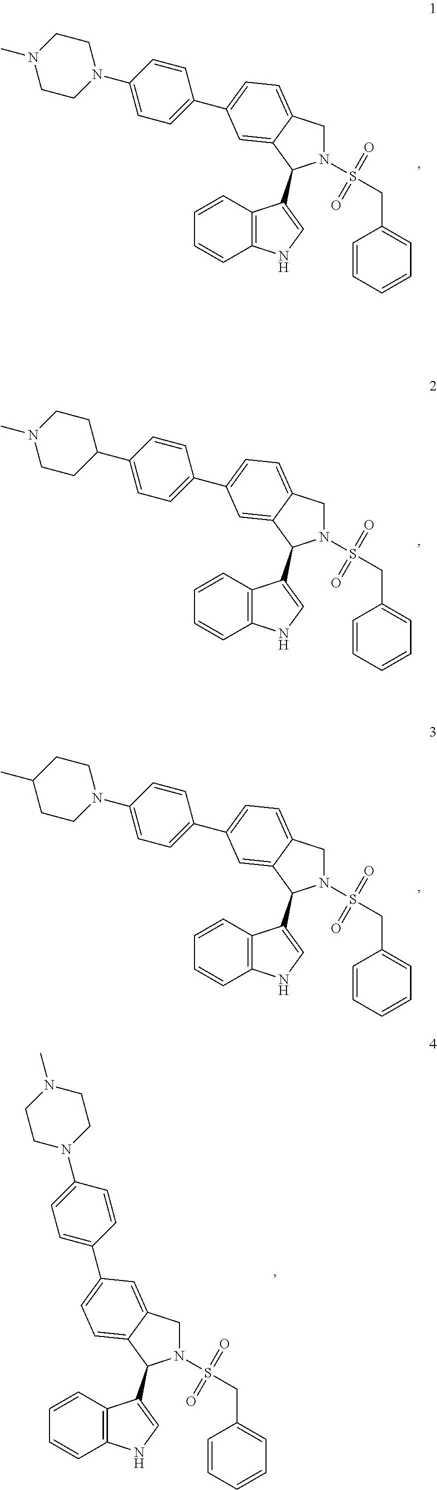 [SMILES: CN1CCN(c2ccc(-c3ccc4c(c3)[C@H](c3c[nH]c5ccccc35)N(S(=O)(=O)Cc3ccccc3)C4)cc2)CC1 ; CN1CCC(c2ccc(-c3ccc4c(c3)[C@H](c3c[nH]c5ccccc35)N(S(=O)(=O)Cc3ccccc3)C4)cc2)CC1 ; CC1CCN(c2ccc(-c3ccc4c(c3)[C@H](c3c[nH]c5ccccc35)N(S(=O)(=O)Cc3ccccc3)C4)cc2)CC1 ; CN1CCN(c2ccc(-c3ccc4c(c3)CN(S(=O)(=O)Cc3ccccc3)[C@H]4c3c[nH]c4ccccc34)cc2)CC1]

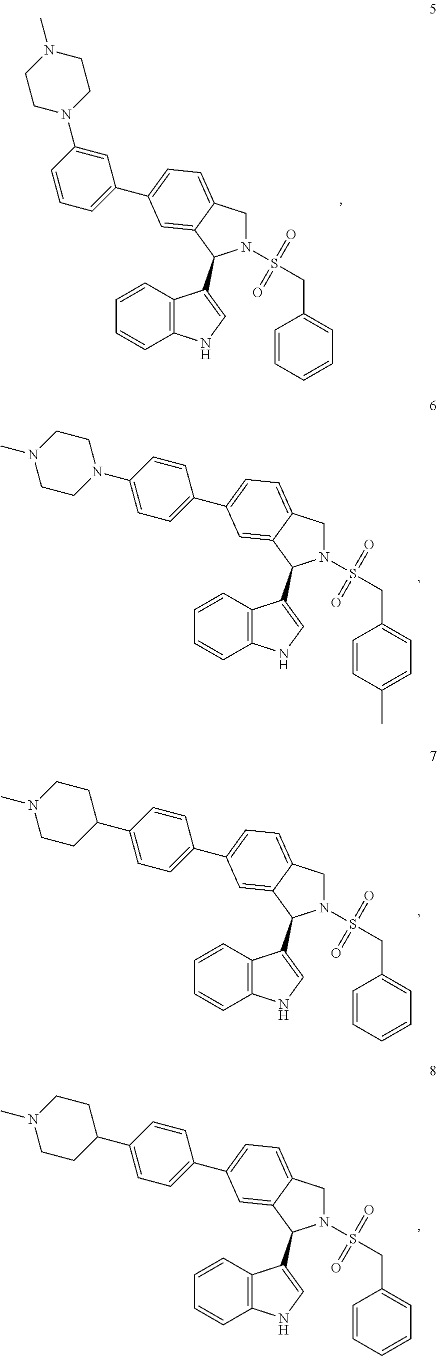 [SMILES: CN1CCN(c2cccc(-c3ccc4c(c3)[C@H](c3c[nH]c5ccccc35)N(S(=O)(=O)Cc3ccccc3)C4)c2)CC1 ; Cc1ccc(CS(=O)(=O)N2Cc3ccc(-c4ccc(N5CCN(C)CC5)cc4)cc3[C@@H]2c2c[nH]c3ccccc23)cc1 ; CN1CCC(c2ccc(-c3ccc4c(c3)[C@H](c3c[nH]c5ccccc35)N(S(=O)(=O)Cc3ccccc3)C4)cc2)CC1 ; CN1CCC(c2ccc(-c3ccc4c(c3)[C@H](c3c[nH]c5ccccc35)N(S(=O)(=O)Cc3ccccc3)C4)cc2)CC1]

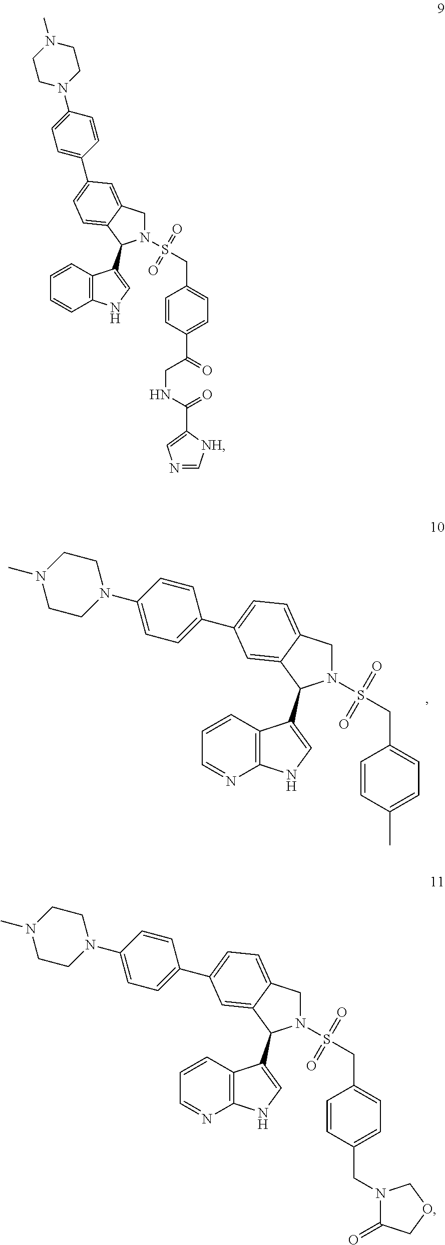 [SMILES: CN1CCN(c2ccc(-c3ccc4c(c3)CN(S(=O)(=O)Cc3ccc(C(=O)CNC(=O)c5cnc[nH]5)cc3)[C@H]4c3c[nH]c4ccccc34)cc2)CC1 ; Cc1ccc(CS(=O)(=O)N2Cc3ccc(-c4ccc(N5CCN(C)CC5)cc4)cc3[C@@H]2c2c[nH]c3ncccc23)cc1 ; CN1CCN(c2ccc(-c3ccc4c(c3)[C@H](c3c[nH]c5ncccc35)N(S(=O)(=O)Cc3ccc(CN5COCC5=O)cc3)C4)cc2)CC1]

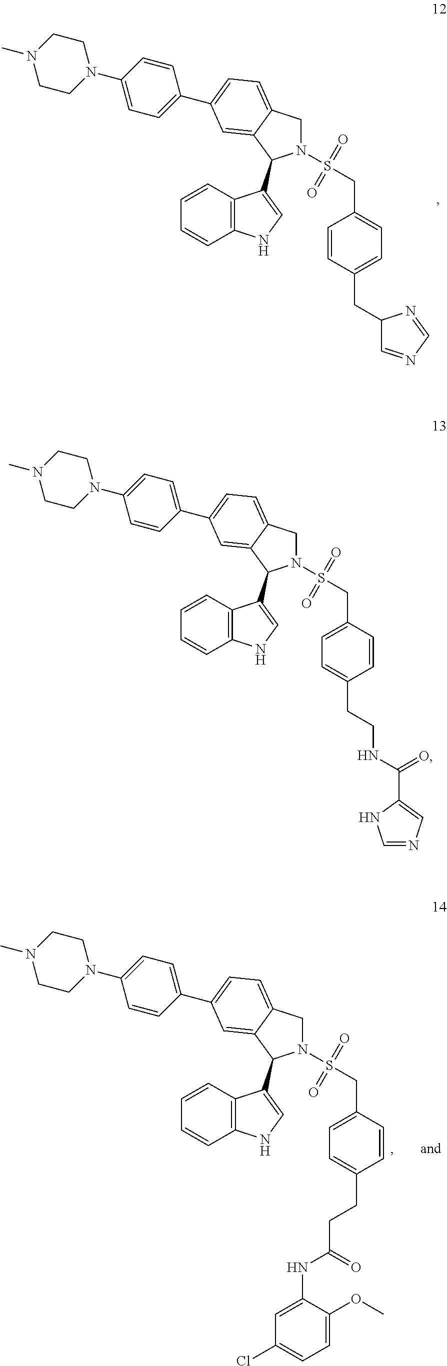

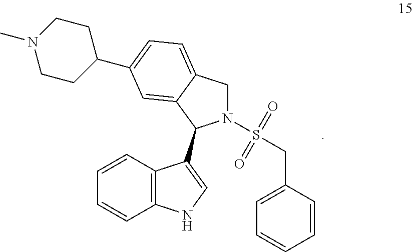 [SMILES: CN1CCC(c2ccc3c(c2)[C@H](c2c[nH]c4ccccc24)N(S(=O)(=O)Cc2ccccc2)C3)CC1]

In one embodiment, the compound of the present invention is Compound 1, JKYN-1 (base), having the formula:

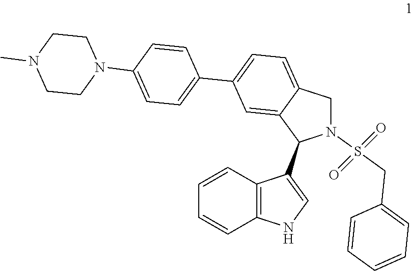 [SMILES: CN1CCN(c2ccc(-c3ccc4c(c3)[C@H](c3c[nH]c5ccccc35)N(S(=O)(=O)Cc3ccccc3)C4)cc2)CC1]

or its mesylate salt Compound 1-mes, JKYN-1-mesylate, having the formula:

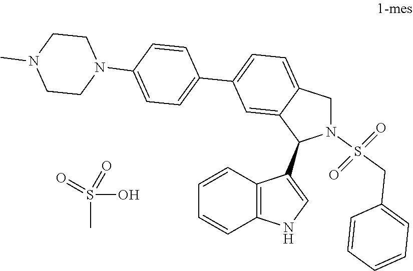

The compounds of the present invention incorporate solubilizing moieties to increase solubility in aqueous solutions. Increased aqueous solubility can be expected to provide therapeutic agents having increased bioavailability. Solubilizing moieties that may be incorporated into the compounds of the present invention include moieties having, for example, multiple hydrogen bonding sites, positively charged moieties, and/or negatively charged moieties.

Increased aqueous solubility can also facilitate the preparation of pharmaceutical formulations.

The present invention also includes novel methods of treating or preventing cancer using the compounds of the invention. In one embodiment, the cancer is selected from the group consisting of breast cancer, chronic myelogenous leukemia, osteosarcoma, glioblastoma, cervical cancer, lung cancer, colon cancer, melanoma, ovarian cancer, prostate cancer, liver cancer, pancreatic cancer, CNS tumors (including brain tumors), neuroblastoma, leukemia, bone cancer, intestinal cancer, lymphoma, and combinations thereof.

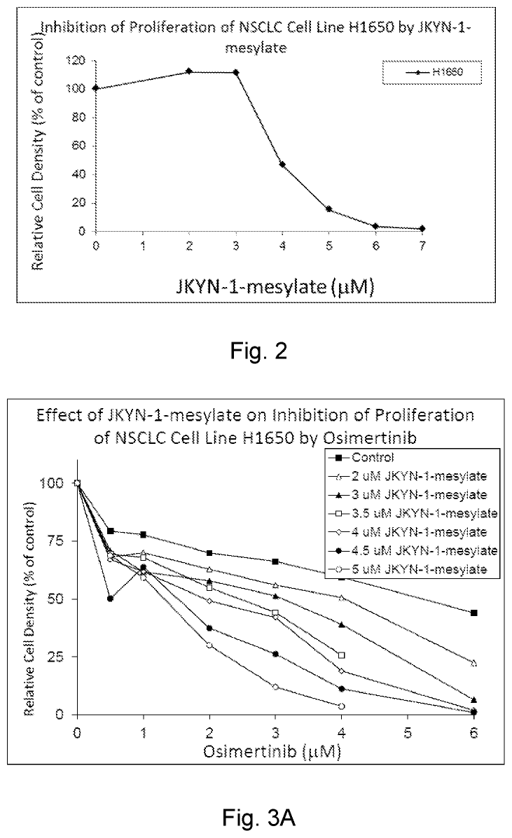

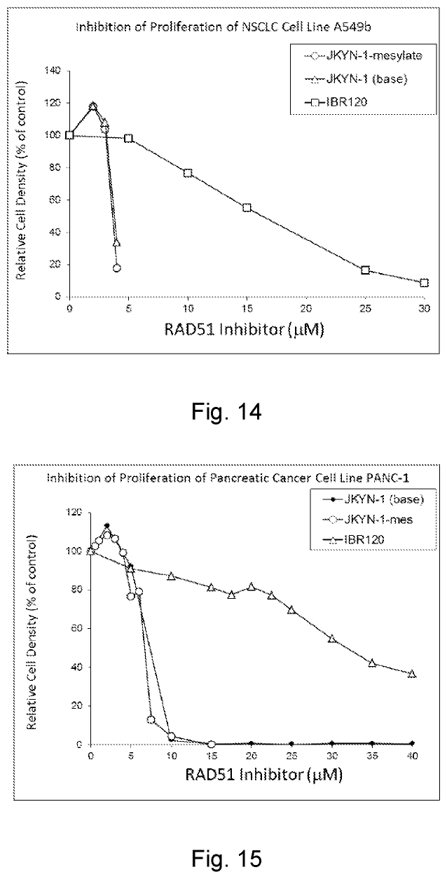

In accordance with one embodiment of the present invention, the compounds of the present invention are for administration as a single therapeutic agent, wherein the anticancer activity is derived from the action of the compound alone. FIGS. 2, 5, 8, 11, 14 and 15 each demonstrate the effectiveness of compounds of the present invention as single therapeutic agents. FIGS. 14 and 15 further demonstrate the improved potency of the compounds of the present invention relative to the prior art compound IBR120.

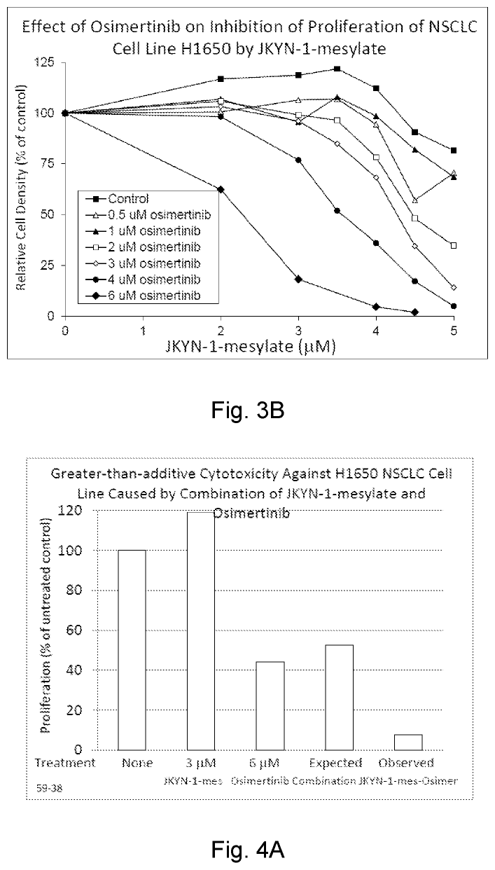

In accordance with another embodiment of the present invention, the compounds of the present invention are used to potentiate another chemotherapy, and are intended for administration in combination or conjunction with a second chemotherapeutic agent or regimen. FIGS. 3A-B, 4A-C, 6A-B, 7A-B, 9A-B, 10A-C, 12A-B, 13, 16A-B, and 17 to 22 each demonstrate the effectiveness of compounds of the present invention as potentiators of known chemotherapeutic agents.

When administered in combination or conjunction with a second chemotherapeutic agent or regimen, the observed potentiation, or enhanced activity, may be attributed to an additive effect, a synergistic effect, or a reversal of resistance.

Where a combination of chemotherapeutic agents is employed, the second therapeutic agent may be any agent that exhibits anticancer activity. Non-limiting examples of such anticancer therapeutic agents that can be potentiated by compounds of the present invention include osimertinib, tamoxifen, enzalutamide and regorafenib.

Osimertinib, for example, is an EGFR-tyrosine kinase inhibitor, and it is contemplated that the compounds of the present invention would similarly potentiate other EGFR-tyrosine kinase inhibitors. In a similar manner, tamoxifen (and 4-hydroxytamoxifen, which is the active metabolite of tamoxifen) is one representative of many anti-estrogen drugs used to treat breast cancers; enzalutamide is one example of an anti-androgen drug used to treat prostate cancer; and regorafenib and afatinib are examples of many multi-targeted kinase inhibitors. Identification of these anticancer agents is not intended to be limiting in scope, and are merely presented as exemplary agents that the compounds of the present invention have been observed to potentiate, and are intended only to exemplify a synergism/additivity paradigm. It is understood that the potentiation activity of the compounds of the present invention can be expected to be observed in combination with other agents not listed here.

In a similar manner, the cell lines selected to demonstrate the synergism/additivity were selected according to the potentiated drugs used in the clinic for the diseases that these cell lines exemplify. For instance, H1650 has an oncogenic EGFR mutation and is inhibited by EGFR inhibitors like osimertinib; MCF7 is an estrogen-dependent line in which tamoxifen is also efficacious; N87 is a stomach cancer cell line that is representative of tumors that may be treated with regorafenib; LNCaP and DU145 are prostate cancer lines that are representative of tumors that may be treated with enzalutamide. Additional cell lines selected for investigation of the synergism/additivity of the compounds of the present invention include PANC-1, which is a pancreatic adenocarcinoma cell line, and A549b, which is a NSCLC cell line. Identification of these cell lines is not intended to be limiting in scope. It is believed that the compounds of the present invention by direct extrapolation would be reasonably expected to work in the clinic.

The synergistic effect observed upon combination of the compounds of the present invention with targeted agents such as osimertinib (in EGFR mutated non-small-cell lung cancer), tamoxifen (in ER+ breast cancer), enzalutamide (in prostate cancer), and regorafenib (in a range of cancers) will allow the use of lower doses of the compounds of the present invention, and so minimize any inherent toxicity effects of the compounds on normal tissues. This would allow the use of lower doses of the compounds of the present invention without compromising the anti-cancer efficacy. The observed synergistic effect can also provide the added benefit of using lower doses of the targeted agent, which is of particular benefit for those agents which have high toxicity profiles, such as regorafenib.

It should be further noted that the single agent activity could occur in any cancer cell line dependent on RAD51, and therefore the use of the compounds as a single therapeutic agent is not limited to the above identified targeted drug synergism examples.

Furthermore, as the understanding of the range of RAD51 mechanisms of action is still incomplete, it should be understood that there is no intent to be limited to a selected mechanism of action as the basis for the observed utility of the compounds of the present invention, except to note that it is believed that RAD51 inhibition plays a role.

As such, the mechanism of action of the compounds of the present invention, either as single agents or as potentiators of other drugs, may relate to DSB repair inhibition but also to inhibition of the other functions of RAD51, including but not limited to, replication fork stabilization and protection.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the term “about” refers to a +/−10% variation from the nominal value. It is to be understood that such a variation is always included in a given value provided herein, whether or not it is specifically referred to.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, methanesulfonic (mesylate), hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

An “effective amount” of a delivery vehicle is that amount sufficient to effectively bind or deliver a compound.

As used herein, the term “alkyl,” by itself or as part of another substituent means, unless otherwise stated, a straight or branched chain hydrocarbon having the number of carbon atoms designated (i.e., C1-6 means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(O)OH, trifluoromethyl, —C(O)O(C₁-C₄)alkyl, —C(O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to: monocyclic cycloalkyls such as cyclopropyl, cyclobutyl, cyclopentyl, cyclohexyl, cycloheptyl, and cyclooctyl; dicyclic cycloalkyls such as tetrahydronaphthyl, indanyl, and tetrahydropentalene; and polycyclic cycloalkyls such as adamantine and norbornane.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups include: non-aromatic heterocycles such as monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “heteroaryl” or “heteroaromatic” refers to a heterocycle having aromatic character. A polycyclic heteroaryl may include one or more rings that are partially saturated. Examples of heteroaryl groups include, but are not limited to, the following: pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(O)₂alkyl, —C(O)NH[substituted or unsubstituted alkyl, or substituted or unsubstituted phenyl], —C(O)N[H or alkyl]₂, —OC(O)N[substituted or unsubstituted alkyl]₂, —NHC(O)NH[substituted or unsubstituted alkyl, or substituted or unsubstituted phenyl], —NHC(O)alkyl, —N[substituted or unsubstituted alkyl]C(O)[substituted or unsubstituted alkyl], —NHC(O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCHYCH₃, —OCH(CH₃)₂, —OCF₃, —OCH₂CF₃, —S(O)₂—CH₃, —C(O)NH₂, —C(═O)—NHCH₃, —NHC(O)NHCH₃, —C(O)CH₃, —ON(O)₂, and —C(O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

The present invention also provides pharmaceutical compositions comprising a compound in accordance with the present invention, and a pharmaceutically acceptable carrier or diluent.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixture of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are sterile, fixed oils which are conventionally employed as a solvent or suspending medium, and a variety of bland fixed oils including, for example, synthetic mono- or diglycerides. In addition, fatty acids such as oleic acid find use in the preparation of injectables.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philadelphia, PA (2000) (formerly “Remingtons Pharmaceutical Sciences”).

The invention will now be described with reference to specific examples. It will be understood that the following examples are intended to describe embodiments of the invention and are not intended to limit the invention in any way.

### EXAMPLES

### Example 1a: Characterization of Compound 1 (JKYN-1, base) ((R)-3-(2-(Benzylsulfonyl)-6-(4-(4-methylpiperazin-1-yl)phenyl)isoindolin-1-yl)-1H-indole)

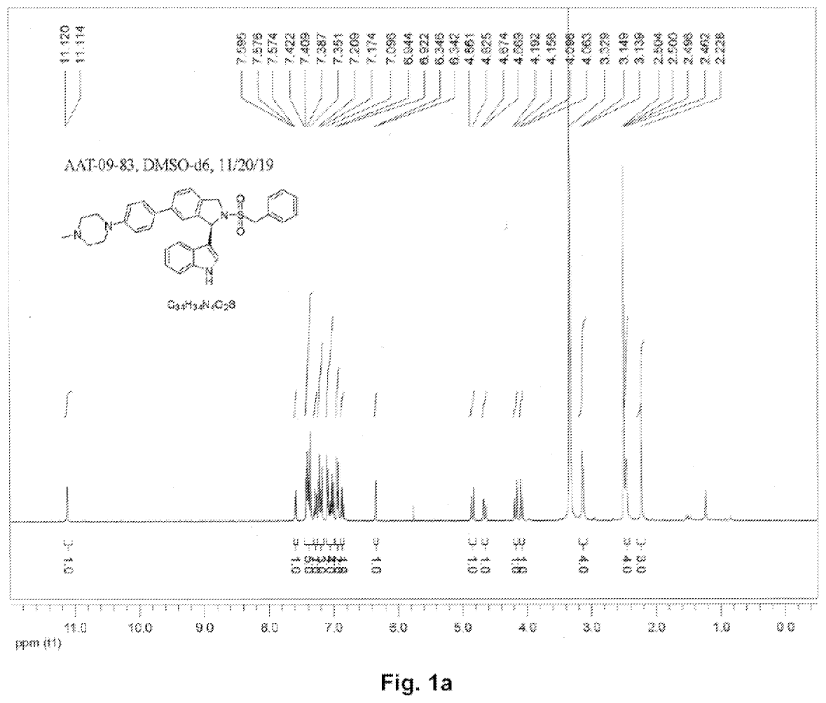

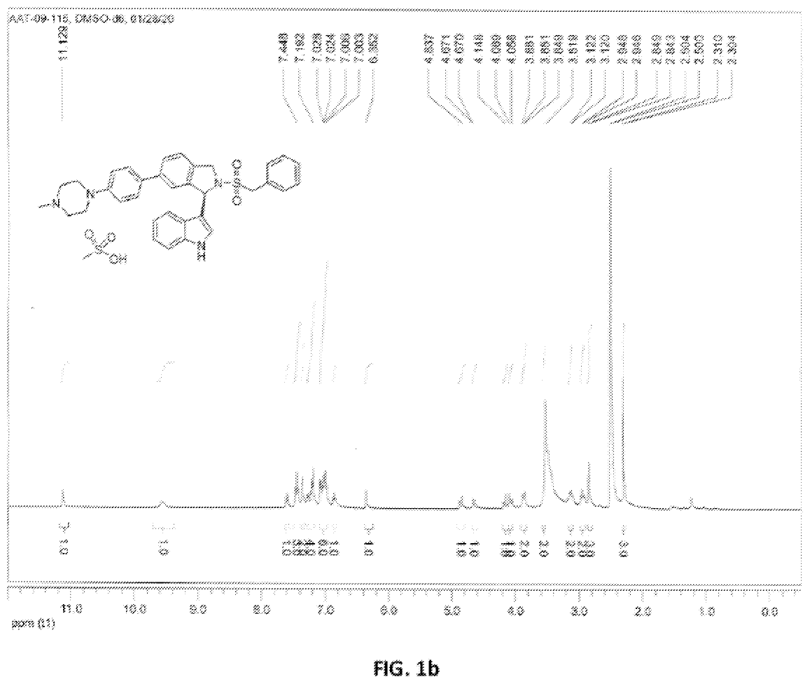

Compound 1 (JKYN-1, base) was synthesized and the structure was characterized by ¹H-NMR and mass spectrometry. The resulting purified compound was a pale yellow solid, having a predicted molecular weight of 562.73 (C₃₄H₃₄N₄O₂S), and observed [M+H]⁺=563.3 (MS[ESI+ve]). FIG. 1a depicts the ¹H-NMR spectrum of Compound 1 (base) obtained in DMSO-d6.

### Example 1b: Characterization of JKYN-1-mes ((R)-3-(2-(Benzylsulfonyl)-6-(4-(4-methylpiperazin-1-yl)phenyl)isoindolin-1-yl)-1H-indole, Mesylate Salt)

JKYN-1-mes was prepared from Compound 1, and the structure was characterized by ¹H-NMR. The resulting purified salt compound was a beige solid. FIG. 1b depicts the ¹H-NMR spectrum of JKYN-1-mes, obtained in DMSO-d6.

### Example 2: Solubility Comparison of RAD51 Inhibitors

A comparison of the solubility of selected compounds of the present invention with known RAD51 inhibitors was carried out.

It was observed that the JKYN-1-mes (the mesylate salt of Compound 1) (MW 658.83) was highly soluble in water compared to the free base. Aqueous samples of the mesylate salt were readily prepared and were suitable for testing in inhibition assays. Exemplary solutions were prepared as follows:

soluble in water−1.0 mg JKYN-1-mes+75.9 μl of water=20 mM (13.2 mg/ml)

50.00 μl of 20 mM+50.0 μl water=10 mM.

Thus, JKYN-1-mes was observed to be completely soluble in water to at least 20 mM. The upper limit of solubility was not determined.

Compound 1 (JKYN-1, free base) was not very soluble in water at 10 mg/ml, and required presolubilization in DMSO prior to dilution in water for testing in inhibition assays. Exemplary solutions were prepared as follows:

1.0 mg Compound 1+88.9 μl of DMSO=20 mM (11.2 mg/ml)

3.00 μl of 20 mM+57 μl water=1 mM

This 1 mM solution of Compound 1 in H₂O/DMSO was less cloudy than 1 mM IBR120, suggesting that it was more soluble in a mixture of H₂O/DMSO than IBR120.

In a similar manner, the prior art compound IBR2 (base, MW 400.50) was insoluble in water and soluble in DMSO. IBR2 was soluble in DMSO to at least 166.8 mM (66.7 mg/ml). Exemplary solutions were prepared as follows:

4.00 mg of IBR2 in 1.00 ml DMSO=10 mM

100 μl of 10 mM+900 μl water=1 mM

The 1 mM solution of IBR2 in H₂O/DMSO was slightly cloudy, suggesting that 1 mM is close to the upper limit of solubility of IBR2 in H₂O/DMSO. When diluted 1:10 or more in water, the solution is clear. To minimize the potential for precipitation over time, IBR2 is always stored as a solid or in DMSO exclusively, and never in a water-containing solution.

Similarly, the prior art compound IBR120 (base, MW 388.49; sodium salt C₂₃H₂₀N₂O₂SNa, MW 411.1143) was insoluble in water and soluble in DMSO. IBR120 is soluble in DMSO to at least 20 mM (7.77 mg/ml). Exemplary solutions were prepared as follows:

7.77 mg of IBR120 in 1.00 ml DMSO=20 mM

5.00 μl of 20 mM+95 μl of water=1 mM

The 1 mM solution of IBR120 in H₂O/DMSO was a little bit cloudy. When diluted 1:10 or more in water, the solution is clear. To minimize the potential for precipitation over time, IBR120 is always stored as a solid or in DMSO exclusively, and never in a water-containing solution. A solution of 25 mg/ml of IBR120 in 100% DMSO dissolves well, and can be kept frozen for several weeks. For purpose of in vivo gavage, IBR120 was dissolved in DMSO to generate a 25 mg/ml stock solution as follows:

16 mg IBR120+640. μl DMSO=25 mg/ml

To generate a solution suitable for in vivo gavage of mice, 900 μl of sterile corn oil was added to 100 μl of 25 mg/ml IBR120 in DMSO. Appropriate amounts of the resulting solution (2.5 mg IBR120/ml) were used to gavage mice to assess any possible overt toxicities.

### Example 3: Assay of Effect of RAD51 Inhibitor on Antiproliferative Effect of Chemotherapy Drugs

Proliferation assays: Cells are maintained in minimum essential medium alpha plus 10% fetal bovine serum and penicillin (50 units/mL)/streptomycin (50 mg/L) (growth medium). Cultures are incubated in a humidified atmosphere of 5% CO₂ at 37° C. Rapidly proliferating cells are used to establish cultures of experimental cells, which are allowed to adhere overnight in 96-well plates or 25-cm² flasks prior to manipulation.

Exposure to chemotherapy drugs and combinations thereof: Cells are grown in 96-well plates and treated with simultaneous exposure to a single drug or a combination of two drugs at the concentrations indicated. The relative cell density is determined after 4 days by viability staining (alamarBlue© or neutral red).

The relative density of cells treated with two agents is normalized to the density observed for a respectively treated “second agent” control. The concentration of drug that inhibits proliferation by 50% (IC₅₀ value) is interpolated from plotted data. The change in the IC₅₀ value of a chemotherapy drug caused by the presence of the second agent is determined as a percent of the IC₅₀ of the first drug alone. Enhancement of cytotoxicity is indicated by a decrease in the IC₅₀ value. This methodology, previously reported (Ferguson et al., 2009; Blake et al., 2017; Ferguson et al., 2018), provides a quantitative, concentration-dependent representation of the magnitude of the drug interaction.

### Example 4: Inhibition of Proliferation of Non-Cancerous Cell Lines by JKYN-1 (base) and JKYN-1-mesylate

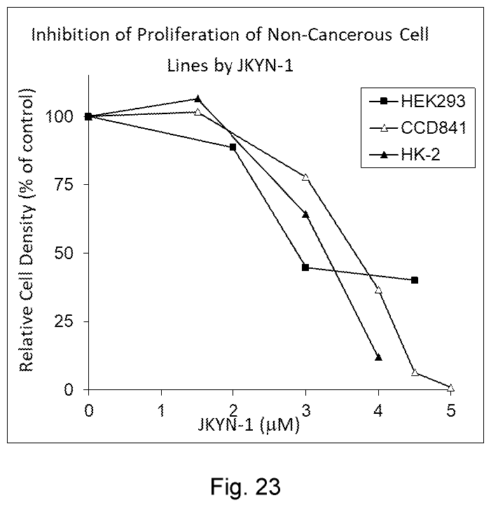

FIG. 23 graphically depicts the toxicity effect on normal (not fully tumorigenic) cell line surrogates resulting from exposure to JKYN-1 (base). The cell line surrogates include: HEK293 (human embryonic kidney transformed with adenovirus 5 DNA); CCD841 (human embryonic colonic epithelial, untransformed); and HK-2 (human kidney proximal tubule, papilloma).

The observed IC₅₀ values for JKYN-1 (base) are as follows: HEK293, 4.16+/−0.77 (n=9); CCD841, 5.71+/−1.97 (n=3); HK-2, 3.92+/−0.32 (n=6). By way of comparison, the observed IC₅₀ values for JKYN-1-mesylate are as follows: HEK293, 2.89+/−0.01 (n=2); CCD841, 3.68 (n=1); HK-2, 3.42+/−0.15 (n=3). From these results, the toxicity of JKYN-1-mes on normal cells appears to be on the order of approximately 3 to 4 μM. From these results, the toxicity of JKYN-1 on normal cells appears to be on the order of approximately 4 to 6 μM. Therefore, the toxicities of JKYN-1 and JKYN-1-mesylate are very similar against these cell lines, as observed with the tumor cell lines (FIGS. 14 and 15).

### Example 5: Inhibition of Various Cancer Cell Lines by JKYN-1-mesylate

The IC₅₀ values for JKYN-1-mesylate against a selection of cell lines are as follows: H1650, 3.95 μM (n=1); LNCaP, 3.36±0.37 μM (n=2); MCF-7, 4.36±0.42 μM (n=2); N87, 2.21±1.13 μM (n=2); PANC-1, 4.69±1.14 μM (n=7); MDA-MB-231, 5.66±0.36 μM (n=3); A549b, 3.96±0.44 μM (n=3).

### Example 6: Inhibition of NSCLC Cell Line H1650 by JKYN-1-mesylate

The inhibitory effect of JKYN-1-mesylate as a single agent on the growth of NSCLC cell line H1650 over a range of concentrations is graphically depicted in FIG. 2.

### Example 7: Inhibition of NSCLC Cell Line H1650 by JKYN-1-mesylate and Osimertinib

FIG. 3A graphically depicts the effect of JKYN-1-mesylate on inhibition by osimertinib of the growth of NSCLC cell line H1650. FIG. 3B graphically depicts the effect of osimertinib on inhibition by JKYN-1-mesylate of the growth of NSCLC cell line H1650.

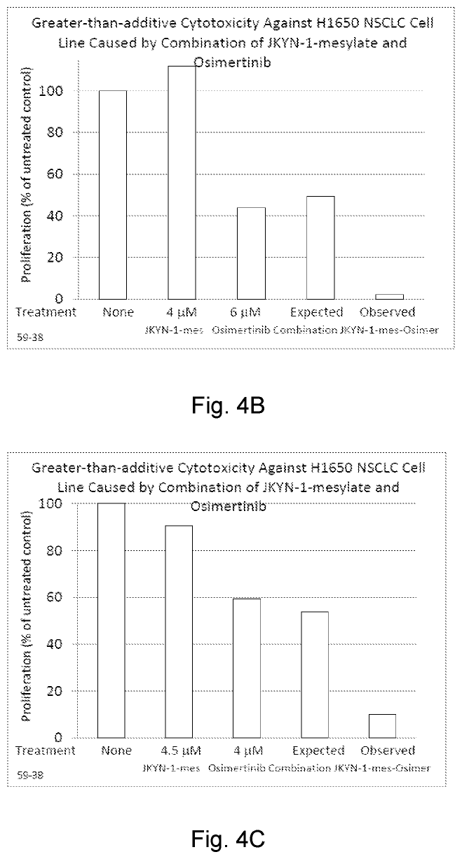

As depicted in FIGS. 4A-C, the combination of JKYN-1-mesylate and osimertinib demonstrates a synergistic effect, in that the combination of the two agents results in a cytotoxic effect that is greater than the expected cumulative effect of the two agents. This synergistic effect is observed over a range of concentrations of the two agents. JKYN-1-mesylate enhanced osimertinib toxicity (decreased the IC₅₀ value of osimertinib) by up to 75%.

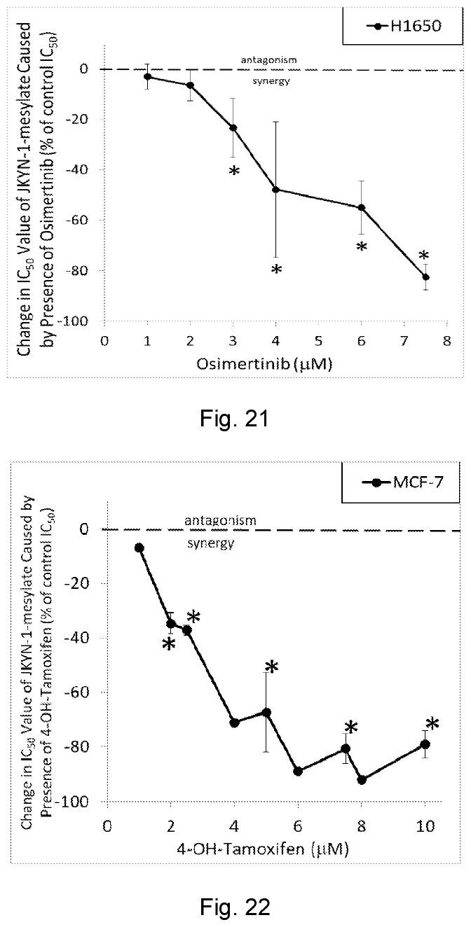

Osimertinib also reduced the IC₅₀ of JKYN-1-mesylate, allowing for the use of lower concentrations of JKYN-1-mesylate while still maintaining an inhibitory effect. This reduction in the IC₅₀ of JKYN-1-mesylate is shown in FIG. 21, which indicates that the IC₅₀ of JKYN-1-mesylate drops from 4 μM or higher (as shown in FIGS. 2 and 3B) down to approximately 2 μM in the presence of 6 μM osimertinib, taking it below the expected toxic IC₅₀ value of approximately 3-4 μM of JKYN-1-mesylate in normal cells (as shown in FIG. 23).

### Example 8: Inhibition of LNCaP Prostate Carcinoma Cell Line by JKYN-1-mesylate

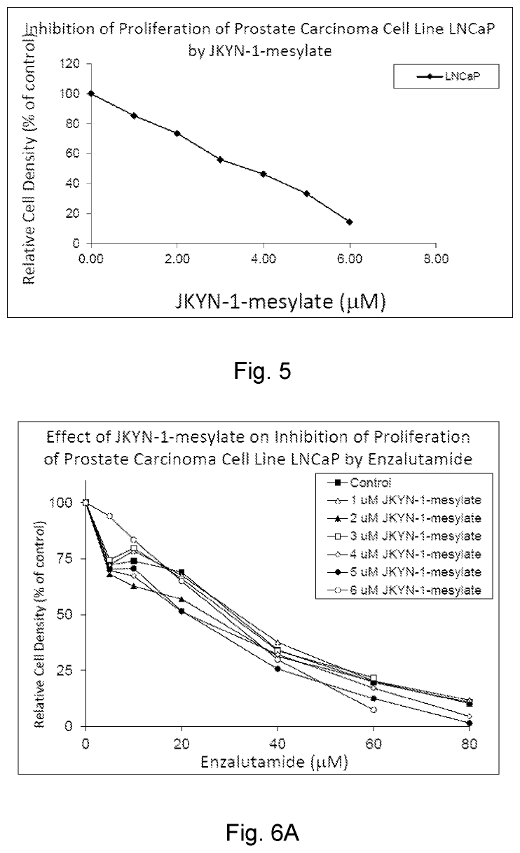

The inhibitory effect of JKYN-1-mesylate on the growth of prostate carcinoma cell line LNCaP over a range of concentrations is graphically depicted in FIG. 5.

### Example 9: Inhibition of LNCaP Prostate Carcinoma Cell Line by JKYN-1-mesylate and Enzalutamide

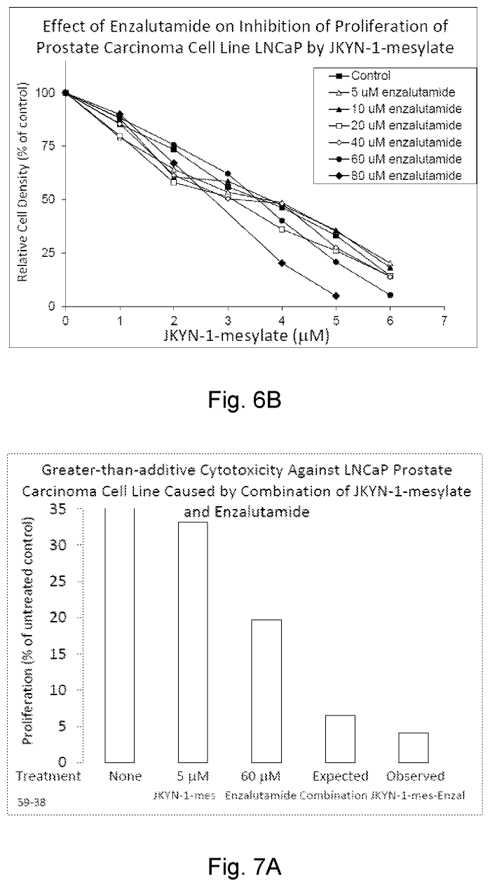

FIG. 6A graphically depicts the effect of JKYN-1-mesylate on inhibition by enzalutamide of the growth of LNCaP prostate carcinoma cell line, and FIG. 6B graphically depicts the effect of enzalutamide on inhibition by JKYN-1-mesylate of the growth of LNCaP prostate carcinoma cell line. JKYN-1-mesylate enhanced enzalutamide toxicity (decreased the IC₅₀ value of enzalutamide) by up to 30%.

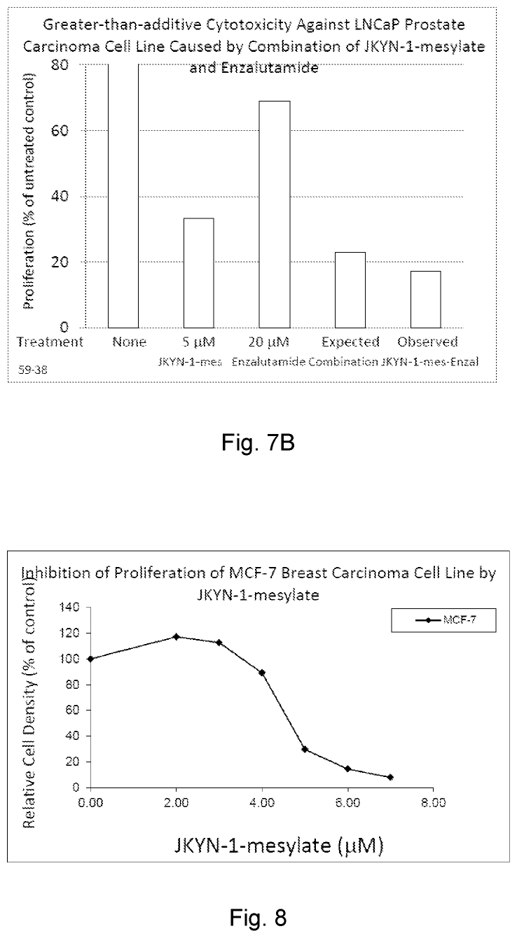

As depicted in FIGS. 7A-B, the combination of JKYN-1-mesylate and enzalutamide demonstrates a synergistic effect, in that the combination of the two agents results in a cytotoxic effect that is greater than the expected cumulative effect of the two agents. This synergistic effect is observed over a range of concentrations of the two agents.

### Example 10: Inhibition of MCF-7 Breast Carcinoma Cell Line by JKYN-1-mesylate

The inhibitory effect of the single agent JKYN-1-mesylate on the growth of MCF-7 breast carcinoma cell line over a range of concentrations is graphically depicted in FIG. 8.

### Example 11: Inhibition of MCF-7 Breast Carcinoma Cell Line by JKYN-1-mesylate and 4-OH-Tamoxifen

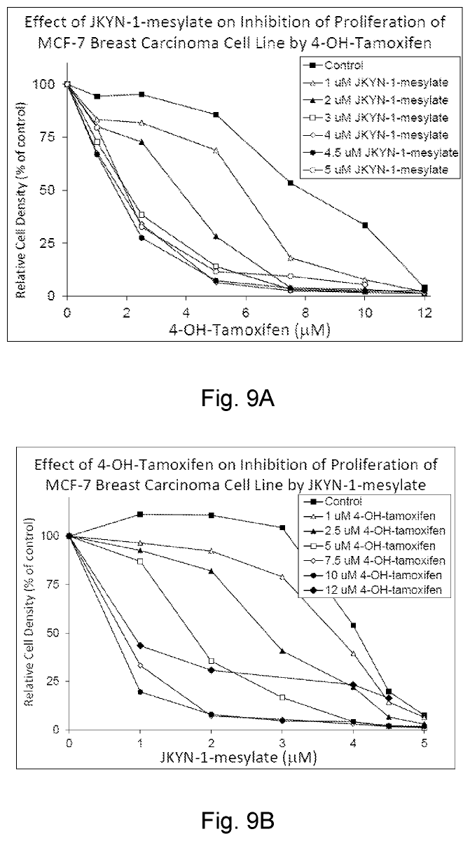

FIG. 9A graphically depicts the effect of JKYN-1-mesylate on inhibition by 4-OH-tamoxifen of the growth of MCF-7 breast carcinoma cell line. FIG. 9B graphically depicts the effect of 4-OH-tamoxifen on inhibition by JKYN-1-mesylate of the growth of MCF-7 breast carcinoma cell line. JKYN-1-mesylate enhanced 4-OH-tamoxifen toxicity (decreased the IC₅₀ value of 4-OH-tamoxifen) by up to 80%.

The use of 4-OH-tamoxifen also reduced the IC₅₀ of JKYN-1-mesylate, allowing for the use of less JKYN-1-mesylate while still maintaining an inhibitory effect. This reduction in the IC₅₀ of JKYN-1-mesylate is shown in FIG. 22, which indicates that the IC₅₀ of JKYN-1-mesylate drops from the 4 to 5 μM range by the addition of about 5 μM (or more) 4-OH-tamoxifen to about 1.5 μM (or less) which suggests there would now be an acceptable therapeutic index between normal and the cancer cells, given the IC₅₀ of JKYN-1-mesylate in normal cell surrogates is about 3-4 μM (as shown in FIG. 23).

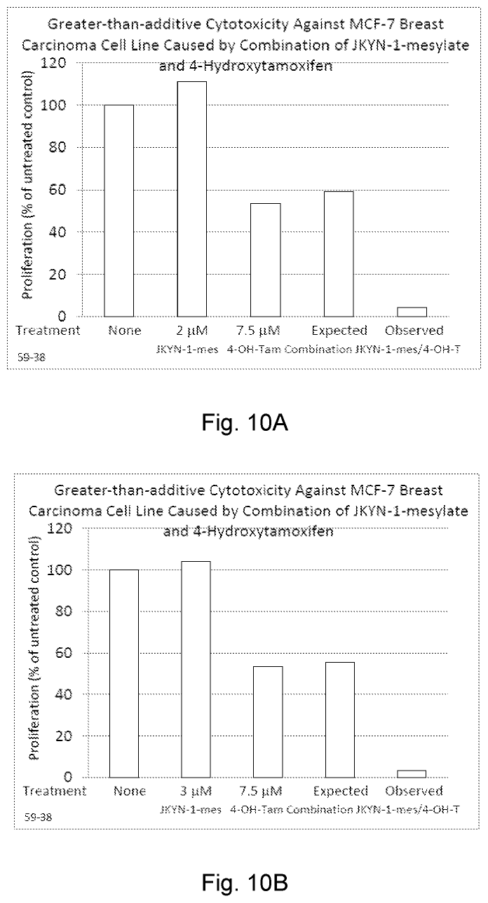

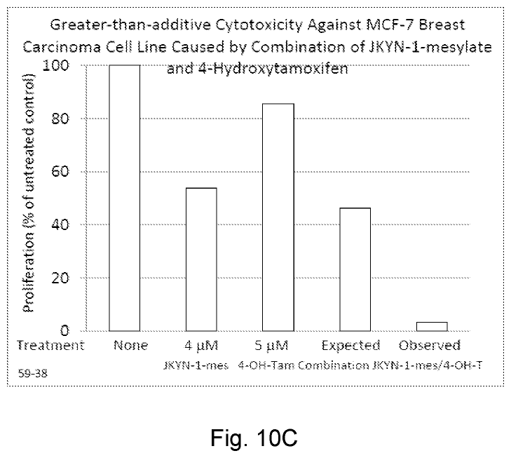

As depicted in FIGS. 10A-C, the combination of JKYN-1-mesylate and 4-OH-tamoxifen demonstrates a synergistic effect, in that the combination of the two agents results in a cytotoxic effect that is greater than the expected cumulative effect of the two agents. This synergistic effect is observed over a range of concentrations of the two agents.

### Example 12: Inhibition of N87 Stomach Carcinoma Cell Line by JKYN-1-mesylate

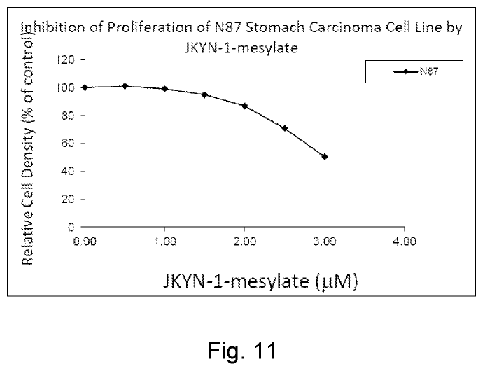

The inhibitory effect of the single agent JKYN-1-mesylate on the growth of N87 stomach carcinoma cell line over a range of concentrations is graphically depicted in FIG. 11.

### Example 13: Inhibition of N87 Stomach Carcinoma Cell Line by JKYN-1-mesylate and Regorafenib

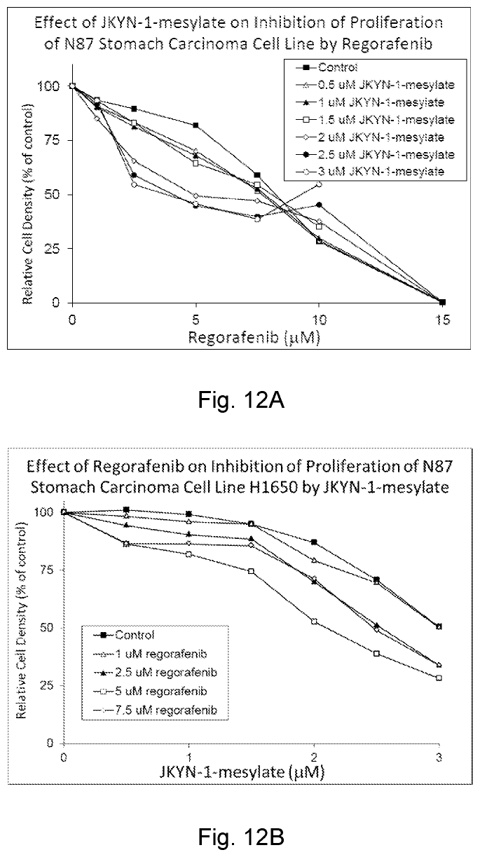

FIG. 12A graphically depicts the effect of JKYN-1-mesylate on inhibition by regorafenib of the growth of N87 stomach carcinoma cell line, and FIG. 12B graphically depicts the effect of regorafenib on inhibition by JKYN-1-mesylate of the growth of N87 stomach carcinoma cell line. JKYN-1-mesylate enhanced regorafenib toxicity (decreased the IC₅₀ value of regorafenib) by up to 54%.

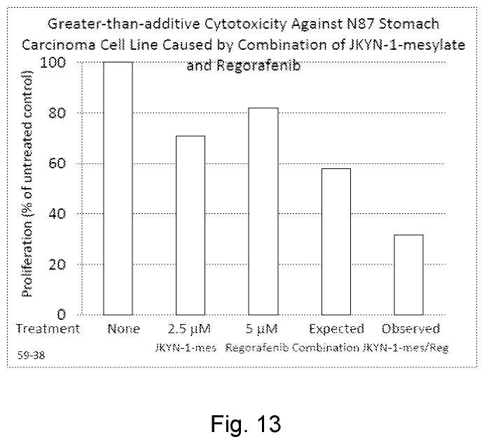

As depicted in FIG. 13, the combination of JKYN-1-mesylate and regorafenib demonstrates a synergistic effect, in that the combination of the two agents results in a cytotoxic effect that is greater than the expected cumulative effect of the two agents.

### Example 14: Comparative Inhibition of NSCLC Cell Line (A549b) Proliferation

FIG. 14 provides a plot of the inhibition NSCLC cell line (A549b) proliferation for each of JKYN-1-mesylate, JKYN-1 (base) and IBR120, over a range of inhibitor concentrations. It is clear that both JKYN-1-mesylate and JKYN-1 (base) have superior activity as compared to the prior art compound IBR120.

### Example 15: Comparative Inhibition of Pancreatic Cancer Cell Line (PANC-1) Proliferation

FIG. 15 provides a plot of the inhibition pancreatic cancer cell line (PANC-1) proliferation for each of JKYN-1-mesylate, JKYN-1 (base) and IBR120, over a range of inhibitor concentrations. It is clear that both JKYN-1-mesylate and JKYN-1 (base) have superior activity as compared to the prior art IBR120.

### Example 16: Inhibition of DU145 Prostate Carcinoma Cell Line by JKYN-1-mesylate and Enzalutamide

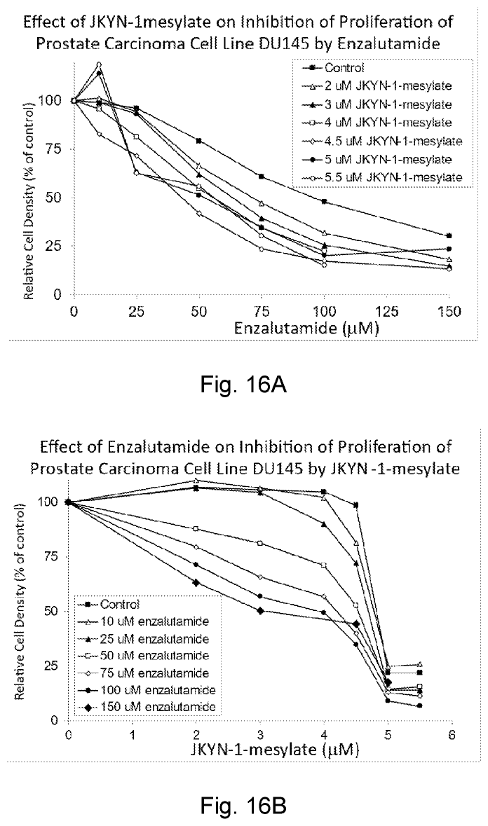

FIG. 16A graphically depicts the effect of JKYN-1-mesylate on inhibition by enzalutamide of the growth of DU145 prostate carcinoma cell line, demonstrating that JKYN-1-mesylate enhanced enzalutamide toxicity.

FIG. 16B graphically depicts the effect of enzalutamide on inhibition by JKYN-1-mesylate of the growth of DU145 prostate carcinoma cell line, demonstrating that, in the presence of enzalutamide, the IC₅₀ of JKYN-1-mesylate is reduced, and less JKYN-1-mesylate is required to kill the cells.

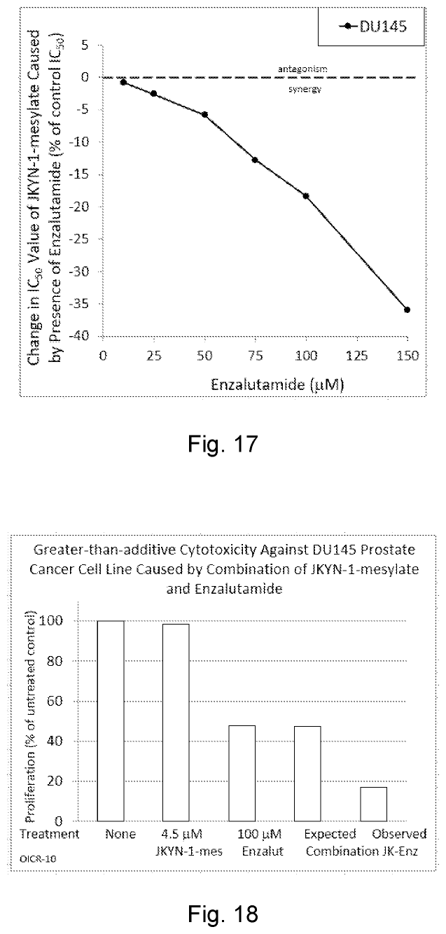

FIG. 17 graphically depicts the synergistic effect of enzalutamide in reducing the IC₅₀ of JKYN-1-mesylate, which indicates that less JKYN-1-mesylate is required to produce an observable inhibitory effect on the cancer cells, which leads to a reduced toxic effect that would result from exposure of normal cells to JKYN-1-mesylate.

As depicted in FIG. 18, the combination of JKYN-1-mesylate and enzalutamide demonstrates a synergistic effect, in that the combination of the two agents results in a cytotoxic effect that is greater than the expected cumulative effect of the two agents. This synergistic effect is observed over a range of concentrations of the two agents. enzalutamide and JKYN-1-mesylate synergy in a prostate cancer cell line, DU145

### Example 18: Inhibition of NSCLC Cell Line (A549b) by JKYN-1-mesylate and Regorafenib

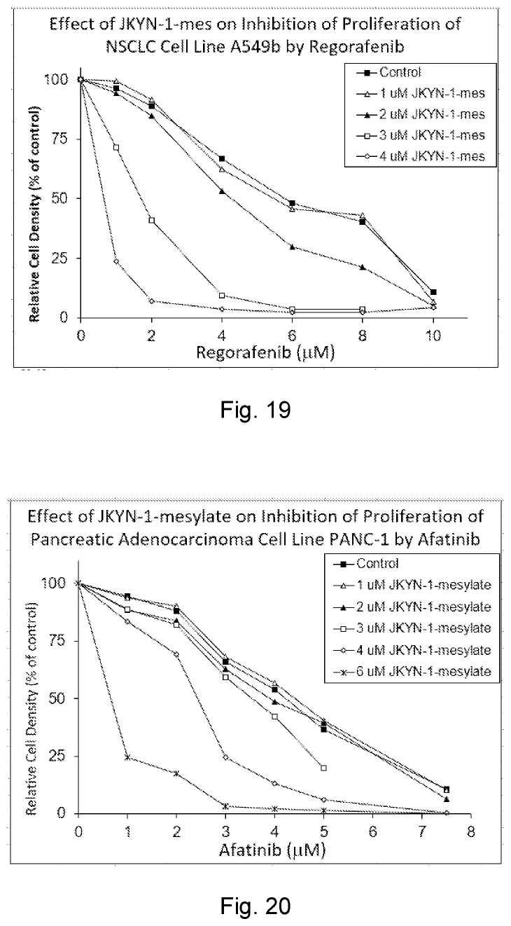

FIG. 19 graphically depicts the effect of JKYN-1-mesylate on inhibition by regorafenib of the growth of NSCLC cell line A549b, demonstrating that JKYN-1-mesylate potentiates regorafenib in a lung cancer line, which is not a usual site for regorafenib, but which may open up a new opportunity for the use of regorafenib in treating lung cancer patients.

### Example 19: Inhibition of Pancreatic Cancer Cell Line (PANC-1) by JKYN-1-mesylate and Afatinib

FIG. 20 graphically depicts the effect of JKYN-1-mesylate on inhibition by afatinib of the growth of pancreatic adenocarcinoma cell line PANC-1. The observed positive (synergistic) effect of JKYN-1-mesylate with afatinib opens up the possible use of afatinib for treatment of pancreatic cancer, which is not a recognized use.

1. A compound having the structural formula (I): 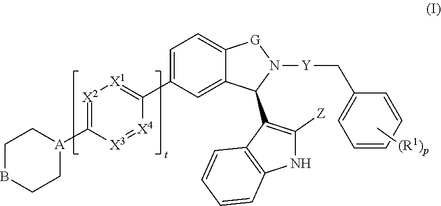
or a pharmaceutically acceptable salt or solvate thereof, wherein:
A is N or CH;
B is CH₂, O, or NR^{a}, wherein R^{a} is —H or C₁₋₄ alkyl;
X¹, X², X³ and X⁴ are each independently N or CR;
t is 0 or 1;
G is —CH₂—, —CHOH—, —CH═CH—, —COH═CH—, or —CH═COH—;
Z is H or OH;
R is independently hydrogen, halogen, hydroxyl, C₁₋₆ alkyl, C₁₋₆ alkoxyl, or N(R^{b})₂, wherein R^{b} is —H or C₁₋₆ alkyl;
Y is —C(O)— or —SO₂—;
R¹ is —(CH)ₙX—R²;
p is an integer from 0-5;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the cycloalkyl group, heterocyclic group, heteroaromatic group, and aryl group are optionally substituted with one or more substituents selected from halogen, hydroxyl, oxo, and lower alkoxyl,
with the proviso that at least one of A is N and B is NR^{a}.

2. A compound according to claim 1, having the structural formula (Ia): 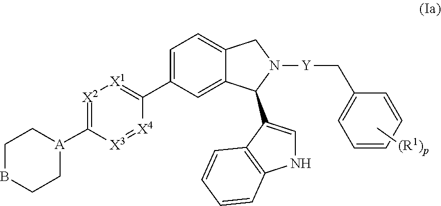
or a pharmaceutically acceptable salt or solvate thereof, wherein:
A is N or CH;
B is CH₂, O, or NR^{a}, wherein R^{a} is —H or C₁₋₆ alkyl;
X¹, X², X³ and X⁴ are each independently N or CR;
R is independently hydrogen, halogen, hydroxyl, C₁₋₆ alkyl, C₁₋₆ alkoxyl, or N(R^{b})₂, wherein R^{b} is —H or C₁₋₆ alkyl;
Y is —C(O)— or —SO₂—;
R¹ is —(CH)ₙX—R²;
p is an integer from 0-5;
n is an integer from 0-6;
X is absent, —C(O)—, —NHC(O)—, —NHC(O)CH₂—, —C(O)CH₂NHC(O)— or —C(O)NH—; and
R² is hydrogen, a cycloalkyl group, a heterocyclic group, a heteroaromatic group, or an aryl group, wherein the cycloalkyl group, heterocyclic group, heteroaromatic group, and aryl group are optionally substituted with one or more substituents selected from halogen, hydroxyl, oxo, and lower alkoxyl.

3. A compound according to claim 1, having the structural formula (IV): 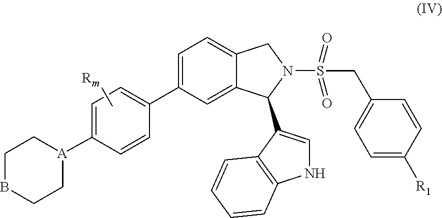
or a pharmaceutically acceptable salt or solvate thereof, wherein:
A is N or CH;
B is CH₂, O, or NR^{a}, wherein R^{a} is —H or C₁₋₄ alkyl;
R is independently hydrogen, halogen, hydroxyl, C₁₋₆ alkyl, C₁₋₆ alkoxyl, or N(R^{b})₂, wherein R^{b} is —H or C₁₋₆ alkyl;
m is an integer from 0-4;
R₁ is —(CH)ₙX—R²;
n is an integer from 0-6;
X is absent, —C(O)—, —NHC(O)—, —NHC(O)CH₂—, —C(O)CH₂NHC(O)— or —C(O)NH—,
R² is hydrogen, a cycloalkyl group, a heterocyclic group, a heteroaromatic group, or an aryl group, wherein the heterocyclic group, heteroaromatic group, and aryl group are optionally substituted with one or more substituents selected from halogen, hydroxyl, oxo, and lower alkoxyl.

4. The compound of claim 1, wherein the compound is a mesylate salt.

5. The compound of claim 1, wherein the compound is selected from the group consisting of:

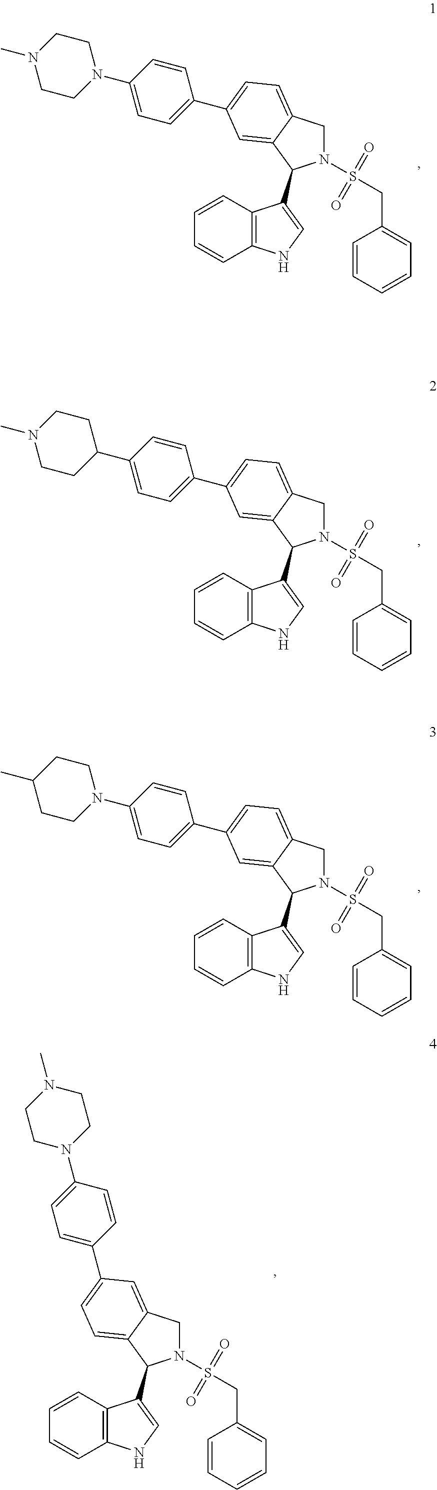 [SMILES: CN1CCN(c2ccc(-c3ccc4c(c3)[C@H](c3c[nH]c5ccccc35)N(S(=O)(=O)Cc3ccccc3)C4)cc2)CC1 ; CN1CCC(c2ccc(-c3ccc4c(c3)[C@H](c3c[nH]c5ccccc35)N(S(=O)(=O)Cc3ccccc3)C4)cc2)CC1 ; CC1CCN(c2ccc(-c3ccc4c(c3)[C@H](c3c[nH]c5ccccc35)N(S(=O)(=O)Cc3ccccc3)C4)cc2)CC1 ; CN1CCN(c2ccc(-c3ccc4c(c3)CN(S(=O)(=O)Cc3ccccc3)[C@H]4c3c[nH]c4ccccc34)cc2)CC1]

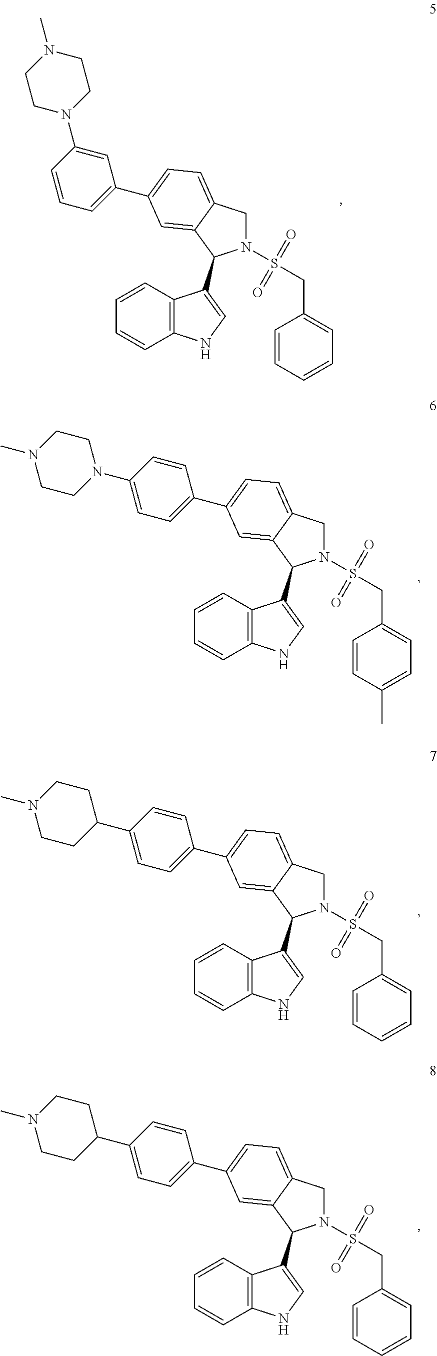 [SMILES: CN1CCN(c2cccc(-c3ccc4c(c3)[C@H](c3c[nH]c5ccccc35)N(S(=O)(=O)Cc3ccccc3)C4)c2)CC1 ; Cc1ccc(CS(=O)(=O)N2Cc3ccc(-c4ccc(N5CCN(C)CC5)cc4)cc3[C@@H]2c2c[nH]c3ccccc23)cc1 ; CN1CCC(c2ccc(-c3ccc4c(c3)[C@H](c3c[nH]c5ccccc35)N(S(=O)(=O)Cc3ccccc3)C4)cc2)CC1 ; CN1CCC(c2ccc(-c3ccc4c(c3)[C@H](c3c[nH]c5ccccc35)N(S(=O)(=O)Cc3ccccc3)C4)cc2)CC1]

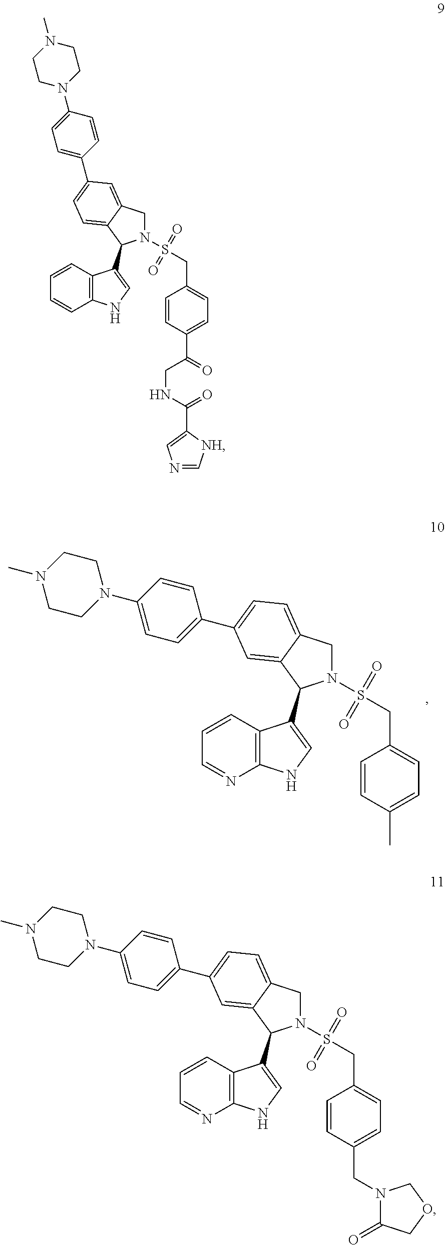 [SMILES: CN1CCN(c2ccc(-c3ccc4c(c3)CN(S(=O)(=O)Cc3ccc(C(=O)CNC(=O)c5cnc[nH]5)cc3)[C@H]4c3c[nH]c4ccccc34)cc2)CC1 ; Cc1ccc(CS(=O)(=O)N2Cc3ccc(-c4ccc(N5CCN(C)CC5)cc4)cc3[C@@H]2c2c[nH]c3ncccc23)cc1 ; CN1CCN(c2ccc(-c3ccc4c(c3)[C@H](c3c[nH]c5ncccc35)N(S(=O)(=O)Cc3ccc(CN5COCC5=O)cc3)C4)cc2)CC1]

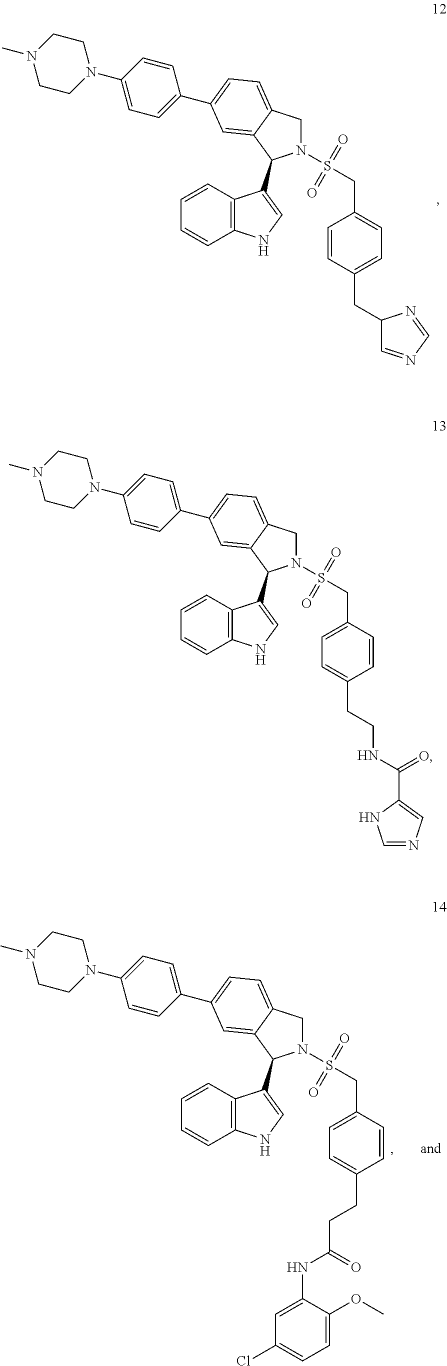

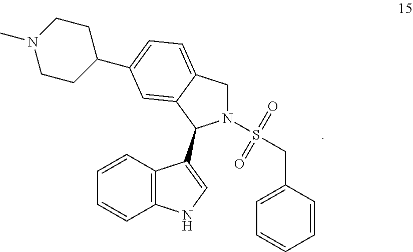 [SMILES: CN1CCC(c2ccc3c(c2)[C@H](c2c[nH]c4ccccc24)N(S(=O)(=O)Cc2ccccc2)C3)CC1]

6. The compound of claim 1, wherein the compound is:

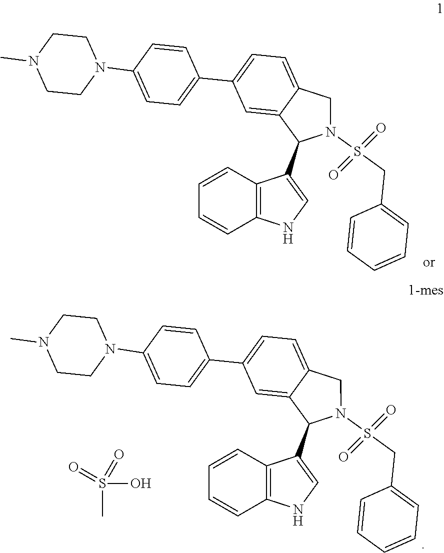

7. A pharmaceutical composition comprising a compound as defined in claim 1, and a pharmaceutically acceptable carrier or diluent.

8. A method of treating or preventing cancer in a subject in need thereof, the method comprising the step of administering a therapeutically effective amount of a compound as defined in claim 1, or a pharmaceutically acceptable salt or solvate thereof, to the subject.

9. A method of treating or preventing cancer in a subject in need thereof, the method comprising the step of administering a therapeutically effective amount of a compound as defined in claim 1, or a pharmaceutically acceptable salt or solvate thereof, in combination with an additional chemotherapeutic agent, to the subject.

10. The method of claim 9, wherein the additional chemotherapeutic agent is one or more of osimertinib, enzalutamide, tamoxifen, afatinib, and regorafenib.

11.-16. (canceled)

17. A pharmaceutical composition comprising a compound as defined in claim 6, and a pharmaceutically acceptable carrier or diluent.

18. A method of treating or preventing cancer in a subject in need thereof, the method comprising the step of administering a therapeutically effective amount of a compound as defined in claim 6, or a pharmaceutically acceptable salt or solvate thereof, to the subject.

19. A method of treating or preventing cancer in a subject in need thereof, the method comprising the step of administering a therapeutically effective amount of a compound as defined in claim 6, or a pharmaceutically acceptable salt or solvate thereof, in combination with an additional chemotherapeutic agent, to the subject.

20. The method of claim 19, wherein the additional chemotherapeutic agent is one or more of osimertinib, enzalutamide, tamoxifen, afatinib, and regorafenib.
